(19) United States
(12) Patent Application Publication
(10) Pub. No.: US 2002/0136694 A1
(43) Pub. Date: Sep. 26, 2002

(54) Method and device for improved two-phase flow through a lumen

(76) Inventors: Randal O. Wallingford, Temecula, CA (US); Jack A. Brown, Sunnyvale, CA (US); Harm TenHoff, Mountain View, CA (US); Craig Coombs, Palo Alto, CA (US)

Correspondence Address: FLEHR HOHBACH TEST ALBRITTON & HERBERT LLP, Four Embarcadero Center, Suite 3400, San Francisco, CA, 94111-4187, US

(21) Appl. No.: 10/013,426

(22) Filed: Dec. 11, 2001

Related U.S. Application Data
(60) Provisional application No. 60/255,084, filed on Dec. 12, 2000.

(51) Int. Cl. A61K 49/04; A61K 31/715; A61M 25/00

(52) U.S. Cl. 424/9.45; 514/2; 514/54; 604/523

(57) ABSTRACT

The addition of surfactant additives to facilitate efficient laminar flow through the lumens of certain medical devices, including catheters. The surfactant can be added to an injection medium, or otherwise located within the catheter itself.

### RELATED APPLICATIONS

This application claims priority to Provisional Application Serial No. 60/255,084 filed Dec. 12, 2000, the entire disclosure of which is hereby incorporated by reference.

### FIELD OF THE INVENTION

The present invention relates to improving fluid flow through lumens of catheters and other medical devices, and more particularly to the use of surfactants to improve such flow in lumens of limited diameters

### BACKGROUND OF THE INVENTION

Many conventional catheters operate to provide fluid through longitudinal channels, or lumens, that extend through the catheter shaft. These catheters are used in a wide variety of applications, including, e.g., angiography catheters that deliver radiopaque dye to a patient's vasculature, balloon dilatation catheters that typically deliver a radiopaque dye to a distal balloon that inflates to dilate a blood vessel and/or deploy a stent, and drug-delivery catheters that typically deliver a drug in solution to a target site.

Fluid flow through the lumens of such catheters is typically established by creating a pressure drop between a proximal fluid source, and the distal fluid target site, for example, a location in a patient's vasculature or a distal dilatation balloon of the catheter, or the like. This is often accomplished through the use of an indeflator or injector connected to a manifold at the proximal end of the catheter to provide fluid under pressure to the catheter. In certain applications, it is desirable for the fluid flow to be reversed, i.e., to be directed from the distal to the proximal end of the catheter, requiring a pressure drop from the distal to proximal end of the catheter. A pressure drop can be established in this direction by applying and maintaining a vacuum at the proximal end of the catheter, e.g., through an indeflator attached to the catheter manifold. Catheter applications that require fluid flow in this direction include, for example, drainage catheters and balloon dilatation catheters for the deflation of the balloon once deployed.

In most cases, the most efficient fluid flow through catheter lumens is achieved by establishing and ensuring the prevalence of laminar flow (also known as “Poiseuille flow”) conditions. A variety of conditions can exist that disrupt or disturb laminar flow through the catheter lumens, such as varying cross-sectional lumen shapes, local constrictions in the lumen, as well as the viscosity of the fluid medium itself. The ability of a medium to sustain a laminar flow under given flow conditions (e.g., catheter diameter and flow velocity) is indicated by its Reynolds number. In addition, the presence within the fluid itself of particles, gas-bubbles, or other emulsified fluids, giving rise to so-called two-phase, or multi-phase flow, will also disturb the laminar flow pattern. Lumen constrictions and two-phase flow have been shown to cause seriously decreased flow velocities, including no-flow conditions.

Where efficient fluid flow is compromised it is sometimes possible to achieve improved flow by increasing the effective pressure drop over the catheter. This can be done, for example, by increasing the injection pressure when injecting a fluid at the proximal end of the catheter for delivery at the distal end. However, in cases of reversed flow direction, i.e., from distal to proximal end of a catheter, the pressure drop can be maximized by applying a vacuum at the proximal end. The amount of force applied in such situations is, however, limited by the extent of the vacuum that can be drawn. Even with a near zero vacuum drawn, under standard atmospheric conditions, the maximum pressure drop will be approximately 1 atmosphere.

Depending on the system, it may be more or less difficult to ensure essentially single-phase flow in the whole system. Common efforts to improve the likelihood of single-phase flow include, for example, “prepping” a balloon dilatation catheter prior to deploying the catheter. “Prepping” the catheter involves removing most of the air from the catheter by pulling a (near) vacuum on the proximal side of the catheter. This serves two purposes. First, in the rare case of balloon rupture under very high pressures, hardly any compressible air remains present in the system that would otherwise be subject to rapid expansion, causing harm to a patient. Second, when deflating the balloon under normal operating conditions, the lack of an appreciable amount of air in the system decreases the chances of two-phase flow developing and helps ensure mostly single-phase flow. Such efforts help achieve fast and efficient balloon collapse and low overall deflation times. In the case of angioplasty procedures, fast deflation times are critical as typically the balloon in its inflated condition is blocking blood flow. Thus deflation times of 15-20 seconds are critical to avoid harm to the patient. Inadequate prepping of the catheter, which can give rise to a “lock-up” condition, is very dangerous.

Even with the best prepping technique, residual air cannot always be completely removed from most catheter systems. The problem becomes particularly acute when pursuing the development of low profile catheters having limited diameter lumens. As lumen size decreases, smaller and smaller amounts of residual air remaining in the system can give rise to two-phase flow conditions, hampering performance of the catheter. At the same time, however, there is an ongoing need for further miniaturization of minimally invasive medical devices such as catheters. In general, this will reduce the size of the trauma to the body to create an entry-port and consequently reduce the healing time. It also allows for simultaneous use of more than one device through the same entry-port and within the same anatomical space limitations to conduct more complex procedures. Particularly, in the case of catheters, such miniaturization necessarily results in increasing smaller diameter lumens for fluid flow. Consequently, as the available space for flow lumens decreases, sensitivity to reduced flow caused by two-phase flow and the problems associated therewith increases.

Therefore, there is a need for a way of enhancing, improving and maintaining efficient fluid flow through the lumens of catheters and other medical devices, especially where it is otherwise desirable to use very small diameter lumens.

### SUMMARY OF THE INVENTION

The present invention meets these and other needs and provides for additional of surfactants, in a variety of forms and methods, to medical devices, including catheters, which rely on fluid flow through small diameter lumens. These surfactants, when added to a fluid medium, can reduce the surface tension between the base medium and any second phase medium present therein, for example, air or an emulsified fluid. The added surfactant results in any air bubbles or emulsified fluid droplets becoming smaller and more finely dispersed in the base medium. This in turn promotes and enhances efficient laminar flow of the medium and decreases the deleterious effects of two-phase flow, including the potential for catheter “lock-up”.

In one embodiment of the invention, the surfactant additive can be added directly to the fluid medium, for example, during the manufacture of the medium itself. In catheter systems, such as a medium (often referred to as an “injection medium”) is typically, e.g., an iodine-based radiopaque dye. Alternatively, the surfactant additive can be added to the medium prior to use of the medium with a catheter or other medical device. For example, the surfactant can be directly mixed with the medium prior to injection of the mixture into a catheter.

In another embodiment of the invention, the catheter itself can be treated with the surfactant additive prior to introduction of the fluid medium. For example, the catheter lumen can be coated with the surfactant additive. Alternatively, the surfactant may be present (either in a liquid or encapsulated form) inside the catheter (e.g. in the manifold or the shaft) and be released into solution upon contact with the fluid medium, when the medium is injected into the catheter.

### BRIEF DESCRIPTION OF THE DRAWINGS

FIG. 1 shows the velocity profiles of fluids passing through a lumen under laminar conditions and non-laminar conditions;

FIG. 2A shows a lumen containing fluid where the fluid includes small, dispersed bubbles and is a condition for efficient laminar flow; and

FIG. 2B shows a lumen containing fluid that includes large bubbles spanning the diameter of the lumen that inhibit efficient laminar flow.

### DETAILED DESCRIPTION OF THE INVENTION

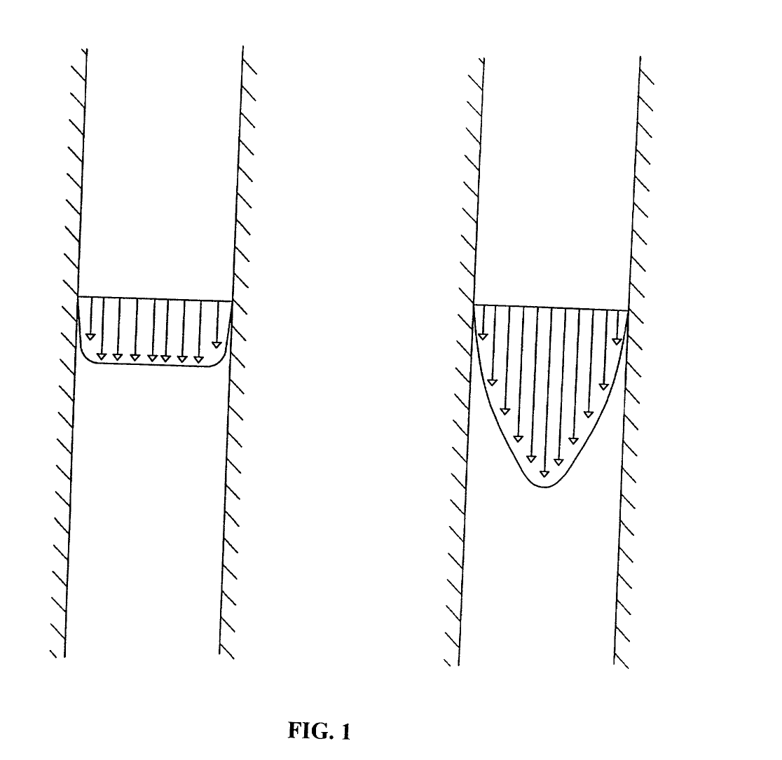

The present invention involves the use of surfactants to improve fluid flow in the lumens of catheters and other medical devices. In particular, the invention aids in establishing and enhancing laminar flow conditions. Efficient laminar flow is characterized by a parabolic flow profile (see FIG. 1). In such a flow profile, maximum flow velocity is achieved by fluid passing through in the lumen center and fluid velocity decreases as the lumen wall is approached, culminating in zero velocity at the lumen wall. This layer at the wall essentially masks any wall unevenness, so that friction at the wall is essentially zero. Flow loss due to frictional losses therefore are those attributable to the viscosity of the fluid itself.

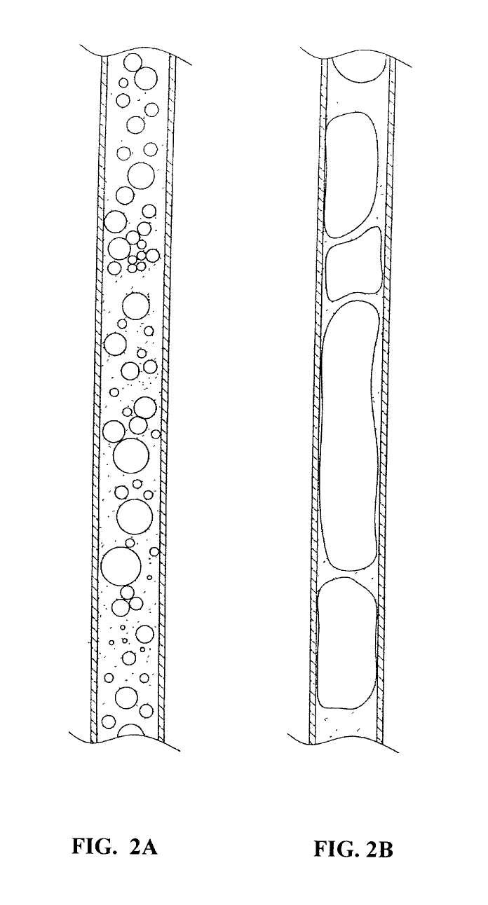

On the other hand, if for example air bubbles are present in the flow medium larger than the lumen diameter, such bubbles will extend across the entire diameter of the lumen, i.e., from wall-to-wall. The result is the creation of a series of gas compartments, i.e. the bubbles, interspersed with areas or compartments of fluid. (FIG. 2B). Laminar flow cannot develop under these conditions. Rather, the velocity of fluid flow under these conditions is virtually constant across the diameter of the lumen, even at the lumen wall (see FIG. 1). Friction along the lumen becomes the dominant force that must be overcome by a pressure drop to produce adequate flow. Even when lumen surfaces are smooth, friction losses in such conditions are higher than under laminar flow conditions at equal average flow velocities.

Additionally, the existence of such flow conditions results in the lumen wall surface becoming intermittently wetted and dried as the sequential fluid and gas compartments pass by a localized wall surface. This phenomenon of intermittent drying and re-wetting of the catheter wall has further implications with respect to the forces required to maintain adequate fluid flow. Most catheters are formed of a polymeric material, metals, or combinations of both. Representative polymeric materials include nylons, polyether block amides, polyurethanes, polyvinyl chlorides, polyimides, polyethylene terephthalates (PET), polyketones, polyurethaneureas and fluoropolymers. Representative metals and metal alloys include stainless steel, platinum, tungsten and Ni-Ti shape memory alloys. When such materials are relatively hydrophobic and the injection fluid water-based, re-wetting of the hydrophobic surface requires energy and therefore hampers flow. On the other hand, when the catheter material is relatively hydrophilic, re-drying of the hydrophilic surface also requires energy, slowing down flow as well.

As used herein, the term “surfactant” or “surface-active agent” shall refer to any substance that, when added to a liquid, reduces its surface tension. Surfactants or surface-active agents are at least partly hydrophilic (water-soluble) and partly lipophilic (soluble in lipids, or oils), and tend to concentrate at the interfaces between bodies or droplets of water and those of oil, or lipids, to act as an emulsifying agent, or foaming agent. Detergents are just one example of surfactants, as are the surface-agents used, e.g., in the dyeing of textiles, to help the dye penetrate the fabric evenly. In such an application, the surface-active agents are used to disperse aqueous suspensions of insoluble dyes and perfumes.

In the context of the present invention, there exist three interfaces in a catheter lumen/fluid system. These three interfaces include a solid/liquid interface (lumen wall and injection fluid), liquid/gas interface (injection fluid and gas bubbles) and gas/solid interface (gas bubbles and lumen wall). There can, of course, be points where all three interact, i.e., an interface between the lumen wall, gas bubble and liquid.

The present invention employs surfactants to reduce the surface tension of the liquid and/or wet the surface of the solid tube and/or reduce the bubble size of the gas present in the tube. Reduced surface tension of the bubbles results in smaller bubble size. By reducing bubble size to less than the diameter of the catheter lumen, two-phase flow is avoided and laminar flow can be achieved (FIG. 2A). As a result “lock-up” phenomena are mitigated if not entirely avoided.

The ability of surfactants to reduce surface tension of a liquid and/or wet the surface of the solid tube and/or to reduce gas bubble size is a general physico-chemical property of surfactants. Thus, most surfactants can thereby promote laminar flow in a catheter (e.g., the aqueous environments typical of dilation catheters).

The composition and construction of the lumen itself will of course have some influence on the two-phase flow. Most devices are made from polymers and/or metals known in the art. Therefore, the amount of surfactant necessary to produce the desired bubble size will vary, depending on lumen composition, size and injection medium characteristics.

In many applications, for example, where leakage of the surfactant into a patient's body could occur, it will be desirable that the surfactant be biocompatible. As used herein, the term “biocompatible” means that the surfactant would not be toxic, harmful, or otherwise cause an adverse reaction in a patient in the amount that could reasonably be anticipated to leak into the patient's system. Suitable biocompatible surfactants include fluorocarbon surfactants, such as those belonging to the PERFLUBRON™ family of surfactants (Alliance Pharmaceutical Corp., San Diego, Calif.), glycols, including polyethylene glycol and propylene glycol, polysaccharides, including hyaluronic acid, and modified protein solutions, including collagen solutions. Other surfactants useful in the invention are partially water-soluble oils and alcohols.

Examples of useful surfactants are listed in Table 1 below.

TABLE 1
Useful Surfactants

| Preferred Surfactants | Other Surfactants |
|---|---|
| Perfluoro family including | Oils (partially water soluble) |
| Perfluoropropane | Linseed Oil |
| Perfluorobutane | Glycerin |
| Perfluoropentane | Ethyl Cellulose |
| Perfluoro-1,-3-dimethylcyclohexane | Polyvinyl Alcohol (PVA) |
| Perfluorodecalin | Poly (Acrylic Acid) |
| Perfluoromethyldecalin | Polysiloxane |
| Perfluoroperhydrofluorene | |
| Perfluoroperhydrophenanthrene | Other Solvents including |
| Glycols including | Isopropyl Alcohol |
| Polyethylene Glycols | |
| Propylene Glycols | |
| Polysaccharides including | |
| Hyaluronic Acid (HA) | |
| Proteins (modified) including | |
| Collagen | |

For catheter applications where biocompatibility is a concern, the preferred surfactant additives are polyethylene glycol and propylene glycol, either neat or in an aqueous solution.

Effective amounts of surfactant that are useful in the invention include any amounts of surfactant capable of preventing a “lock-up” condition. Such amounts can be surprisingly small. As an example, a single drop (approx. 0.065 ml) of a polyethylene glycol solution containing approximately 25-30% by weight polyethylene glycol (PHOTO-FLOW 200™, Eastman Kodak Co., Rochester, N.Y.) into a radiopaque solution was effective in preventing catheter lock-up. Further, a single drop of a 1:200 dilution of the same polyethylene glycol solution was equally as effective.

Due to the extremely small amounts of surfactant needed to produce the desired effect, the surfactant additive can be delivered to catheter systems in numerous ways, each of which has its advantages. For example, the surfactant can be directly mixed with a radiopaque solution or contrast fluid prior to injection or it can be delivered into the lumen or other locations within the catheter where it would mix with a contrast fluid upon introduction of the fluid into the catheter. Other suitable methods include incorporating the surfactant directly into the interior walls of the catheter itself to mix with the fluid and produce the desired effect when the fluid was introduced into the catheter lumen.

Means for incorporating the surfactant additive directly into the catheter include blending the surfactant into the polymer or polymers used to form the catheter during manufacture of the catheter itself, or otherwise fusing, bonding or coating the surfactant to the catheter wall. As an example, a polyethylene glycol can be incorporated into a catheter by blending the polyethylene glycol with the desired polymeric material and co-extruding the blended material to form the catheter shaft.

The surfactant additive can also be provided in an encapsulated form that can then be released under appropriate conditions to produce the desired effect upon contrast fluid within the catheter. As an example, encapsulated surfactant can be located in the manifold area such that the capsule could be broken and surfactant released by mechanical means. Alternatively, the capsule can be formed of, for example, a heat-sensitive material where the surfactant would be released upon a rise in temperature, such as is commonly associated with catheter use due to the body temperature of a patient. Other methods include incorporating the surfactant into, e.g., insoluble materials such as microspheres.

Other means of delivering the surfactant additive into a catheter include incorporating the surfactant into an aerosol that can be sprayed into the catheter lumen. Still other means include formulating the surfactant into a powder form, and adding the powder into the catheter manifold or other locations within the device, such as the lumens or even the balloons.

The invention will be better understood by way of the following examples.

### EXAMPLES

### Example 1

A balloon dilation catheter having a distal lumen size of 0.007 inches at the balloon inflation/deflation port was utilized in this example. The catheter was prepped by attaching an indeflator (ACS) and pulling a vacuum at the proximal end of the catheter to remove air from the system. A radiopaque solution containing 37% iodine (Hypaque-76, Nycomed, Inc., Princeton, N.J., diluted 50-50 with distilled water) was then injected and the balloon inflated to 8 atm. The balloon was retained in a flat horizontal position during inflation. Air bubbles from residual air in the system localized at the distal end of the balloon upon inflation. While maintaining the balloon in the same position, a vacuum was again applied to the catheter to deflate the balloon. The balloon was adequately deflated within an acceptable period of time, within 14-17 seconds.

### Example 2

The catheter of Example 1 was prepped and inflated in the same manner as described in Example 1. Prior to deflation, the balloon was tilted vertically downward, with the distal end below the proximal end of the balloon. Air bubbles from residual air in the system that had been localized at the distal end of the balloon upon inflation migrated upward due to gravitational forces and accumulated at the proximal end of the balloon in the vicinity of the balloon inflation/deflation port. A vacuum was then applied in an attempt to deflate the balloon.

Under these conditions, the balloon did not deflate, causing a “lock-up” condition. It is believed the air bubbles at the deflation port gave rise to a two-phase flow condition in the catheter lumen, with bubbles of a sufficient size and the surface tension at the gas/liquid and gas/solid interfaces such that the drawing force of the vacuum was unable to provide for adequate flow to deflate the balloon. Deflation was finally accomplished by exerting manual pressure on the balloon itself.

### Example 3

The catheter of Examples 1 and 2 was prepped as described in Example 1 with the exception that 1 drop (approximately 0.065 ml) of a solution made of 60-70% water, 25-30% propylene glycol, and 5-10% p-tert-octylphenoxy polyethoxyethyl alcolol (PHOTO-FLOW 200™, Eastman Kodak Co., Rochester, N.Y.) was added to the catheter manifold prior to injecting the radiopaque solution into the catheter lumen to inflate the balloon. As in Example 2, the inflated balloon was then tilted vertically with the distal end of the balloon pointing downward. Air bubbles from residual air in the system again formed at the distal end of the balloon upon inflation and migrated upward to the deflation port. These air bubbles differed markedly on visual observation from the air bubbles observed in Example 2, being generally much smaller. Upon application of vacuum, the balloon deflated rapidly.

### Example 4

Two additional catheters of the same construction as the catheter of Examples 1-3 were prepped as described in Example 1. In this example, 1 drop (approx. 0.065 ml) of PHOTO-FLO 200™ solution (undiluted) was added and mixed directly with the radiopaque solution prior to injection of the solution to inflate the balloon. Again, after inflation, the balloon was tilted vertically with the distal end downward. Air bubbles from residual air in the system again formed at the distal end of the balloon upon inflation and migrated upward to the deflation port. These air bubbles were similar to those observed visually in Example 3, and again were generally much smaller than the air bubbles observed in Example 2. Upon application of vacuum, the balloon of each catheter deflated rapidly, within 14-17 seconds from application of the vacuum.

### Example 5

A catheter of the same construction as the catheter of Examples 1-3 was prepped as described in Example 1. In this example, 1 drop (approx. 0.065 ml) of medical grade propylene glycol (VWR, West Chester, Pa.) was mixed directly with the radiopaque solution prior to injection to inflate the balloon. Again, after inflation, the balloon was tilted vertically with the distal end downward. Air bubbles from residual air in the system again formed at the distal end of the balloon upon inflation and migrated upward to the deflation port. These air bubbles were similar to those observed visually in Examples 3 and 4, and again were generally much smaller than the air bubbles observed in Example 2. Upon application of vacuum, the balloon deflated rapidly, within 14-17 seconds from application of the vacuum.

The specific embodiments described in the specification, including the above Examples, are not intended to limit the scope of the invention, but are only meant to provide illustrative examples within the spirit and scope of the invention. While particular embodiments of the subject invention have been described, it would be obvious to those skilled in the art that various changes and modifications to the subject invention can be made without departing from the spirit and scope of the invention. All such modifications are within the scope of this invention.

We claim:

1. A fluid medium for passage through a lumen of a catheter or other medical device, said medium comprising a biocompatible surfactant.

2. The medium of claim 1 wherein said medium further comprises an emulsion.

3. The medium of claim 1 wherein said biocompatible surfactant is selected from glycols, fluorocarbons, polysaccharides, or proteins.

4. The medium of claim 1 wherein said surfactant comprises propylene glycol or polyethylene glycol.

5. The medium of claim 1 wherein said surfactant comprises propylene glycol.

6. The medium of claim 1 wherein said surfactant comprises ethylene glycol.

7. A medium according to claim 1, said medium further comprising a radiopaque dye.

8. The medium of claim 7 wherein said radiopaque dye further comprises iodine.

9. A catheter or other medical device comprising
a longitudinally extending tube or shaft having a lumen for passage of a liquid medium, and
a biocompatible surfactant in an amount sufficient to substantially decrease the surface tension of a liquid passing through said lumen.

10. The catheter or device of claim 9 wherein said surfactant is a liquid contained in said lumen.

11. The catheter or device of claim 9 wherein said surfactant is a solid contained in said lumen.

12. The catheter or device of claim 9 wherein said surfactant is coated on the lumen wall.

13. The catheter or device of claim 9, wherein said surfactant is incorporated into the material forming the body of said tubing or shaft and is released into said lumen therefrom.

14. The catheter of claim 9 wherein said liquid surfactant is encapsulated.

15. The catheter of claim 9 wherein said biocompatible surfactant is selected from glycols, fluorocarbons, polysaccharides, or proteins.

16. The catheter of claim 9 wherein said surfactant is ethylene glycol.

17. The catheter of claim 9 wherein said surfactant is propylene glycol.

18. A method for reducing resistance to the flow of a fluid through a catheter, said method comprising selecting a biocompatible surfactant and adding said surfactant to said liquid in an amount sufficient to substantially reduce the surface tension of said liquid.

19. A method according to claim 18, wherein said surfactant is selected from the group consisting of fluorocarbons, polyethylene glycols, propylene glycols, fatty acids, proteins, or polysaccharides.

20. A system for transport of a liquid medium, said system comprising:
a catheter providing a lumen for passage of said medium;
a biocompatible surfactant;
means for adding said surfactant to said medium in an amount sufficient to substantially reduce the surface tension of said liquid medium and thereby to substantially reduce resistance to said passage.

21. A system according to claim 20, wherein said surfactant is selected from the group consisting of fluorocarbons, polyethylene glycols, propylene glycols, fatty acids, proteins, or polysaccharides.

22. A system according to claim 20 further comprising a balloon connected to said catheter whereby said balloon is inflated and deflated by said passage.

23. A system according to claim 20 further comprising a manifold in fluid connection with said lumen, said manifold containing said biochemical surfactant.

24. A system according to claim 22, wherein said medium further comprises a radiopaque dye.

25. A system according to claim 20, wherein said surfactant is a polyethylene glycol or a fluorocarbon.

26. A system according to claim 20, wherein said catheter has an inner diameter of about 0.007 inches.

27. A system according to claim 20, wherein said catheter has an inner diameter of at least 0.007 inches.

28. A system according to claim 20, wherein said catheter has an inner diameter of about 0.002 inches.

29. A system according to claim 22, wherein said surfactant is added in an amount sufficient to allow deflation of said balloon within 20 seconds.

30. A medium according to claim 1, wherein said surfactant is added in an amount sufficient to reduce the size of an air bubble.
